(19) United States
(12) Patent Application Publication
(10) Pub. No.: US 2006/0069226 A1
(43) Pub. Date: Mar. 30, 2006

(54) Method of producing esters with antimicrobial bioresistant and fungal resistant properties

(76) Inventor: Thomas Daly, Chicago, IL (US)

Correspondence Address: Thomas Daly, House C, 1400 N. North Park Ave, Chicago, IL, 60610, US

(21) Appl. No.: 11/265,600

(22) Filed: Nov. 2, 2005

Related U.S. Application Data
(63) Continuation-in-part of application No. 11/193,776, filed on Jul. 29, 2005.
Application No. 11/193,776 is a continuation of application No. 10/350,928, filed on Jan. 23, 2003, now abandoned.
(60) Provisional application No. 60/625,423, filed on Nov. 4, 2004.

(51) Int. Cl. C08G 18/00; C07C 231/02

(52) U.S. Cl. 528/44; 554/35

(57) ABSTRACT

A process for making esters with bioresistant, fungal resistant and antimicrobial/antifungal properties with reduced color and improved yield. A fatty acid halide, preferably a chloride, or a fatty acid anhydride is reacted with bromo-nitro-propandiol (BNPD) to form an almost clear ester that can be used in commercial applications like metal working fluids where it is desired to prevent degradation by bio-organisms.

This is a continuation in part of copending application Ser. No. 11/193,776 filed Jul. 29, 2005 which is a continuation of application Ser. No. 10/350,928 filed Jan. 23, 2003, now abandoned. This application is also related to and claims priority from provisional application No. 60/625,423 filed Nov. 4, 2004. Application 60/625,423 is hereby incorporated by reference.

This application also incorporates by reference my other patent application Ser. No. 10/603,356 filed Jun. 25, 2003 entitled Esters with Antimicrobial, Bioresistant and Fungal resistant properties.

### BACKGROUND

1. Field of the Invention

The present invention relates to the field of esters and more particularly to esters with bioresistant, fungal resistant and antimicrobial/antifungal properties and the synthesis of said esters with reduced color and improved yield.

2. Description of the Problem solved by the Invention

Due to environmental regulation, the use of tin, mercury, lead, and other heavy metals to control the growth of microbes in organic systems is now prohibited. In particular metal working fluids (MWF) and metal working fluid bases suffer a failure mode when attacked by microbes. The problem is especially acute in water extendable and emulsion MWF systems. The attack of the microbes on the MWF base causes the pH of the system to drop, which destabilizes the emulsion and also increases the corrosion of metal parts that are exposed to the attacked fluid. Aside from the obvious problems that microbes cause in MWFs, operator health issues also arise due to continuous exposure to high levels of bacteria.

Current systems in place include the addition of biocides to the fluid to prevent the bacteria from breaking down the MWF. One common biocide in use is the family of isothiazolinones. This product family is generally hazardous to handle and causes sensitization in many people when exposed repeatedly. The sensitization often takes the form of itching all over the body, or hives when any part is in contact with the isothiazolinone. Additionally, the isothiazolinone family is relatively unstable at the alkaline pH that most MWFs are maintained at. This then requires the operator to add more material on a regular basis. Also, the microbes develop a tolerance to isothiazolinones. This again requires the operator to increase the amount of the isothiazolinone in the system.

A second biocide technology is the use of formaldehyde condensates. These materials are generally hazardous, but do not lead to sensitization of the operators in contact with the MWF. The formaldehyde condensates do contribute to free formaldehyde in the workplace, but the results are not consistent as to how much formaldehyde they contribute to the workplace atmosphere. Most formaldehyde condensates are volatile and evaporate. This requires their replenishment on a regular basis even when they are not consumed.

What is needed is a system that uses an ester as the MWF base that is not susceptible to microbial attack. The material fails to act as a food source for the microbes that are able to digest the current MWF bases.

The esters that are described in the prior art produce materials that are dark in color. The color produces problems in the desired applications because color is used to gauge the quality of the metal working fluid. Also, if the material is to be used as a monomer or otherwise a component for a polymer, the darker color reduces significantly the application of the finished product. The present invention solves this problem by providing a method of synthesizing in improved yield and reduced color.

### SUMMARY OF THE INVENTION

The present invention relates to the synthesis of an ester that contains an antimicrobial moiety that is linked into the backbone of the molecule. This moiety is, in general, a bromine atom and a nitro (NO2) group linked to one or more of the carbon atoms forming the backbone of the molecule that is the MWF base. The moiety can appear in the backbone of the MWF base in various levels of occurrence. A preferred occurrence of around 1000 parts per million on a weight basis is effective: however the frequency of occurrence can be as low as 5 parts per million to as high as 99-100%. MWF base types within the scope of the invention include, but are not limited to ester, amide, and carbonate, linkages.

It is well known in the art to combine a carboxylic acid and an alcohol in the presence of a suitable catalyst to form an ester. The present invention teaches a method that adds a bromo-nitro substituted alcohol, diol or polyol to a standard alcohol to be used in the ester synthesis. The proportion of substituted compound used is chosen to yield the desired concentration of the moiety in the final MWF base. A preferred diol for the application is bromonitropropanediol or 2-bromo-2--nitro-propane-1-3-diol or simply BNPD. This particular diol is a solid material with varying degrees of solubility in other alcohols and has proven antimicrobial properties.

In addition, BNPD has been shown to have no tetragenecy (cancer causing effects) and is approved by the CFTA at levels of up to 0.1% for use in cosmetics. BNPD has also been used in baby wipes for its antimicrobial properties.

The fact that the active antimicrobial moiety is covalently linked directly into the backbone of the ester reduces its breakdown at the alkaline pHs required of MWFs. In addition, the moiety is not photo-active or decomposed by sunlight or exposure to mineral salts such as calcium chloride, magnesium hydroxide and sodium chloride as are found in hard and softened water.

Because BNPD is a substituted diol, it is a natural reactant to form part of an ester linkage with a carboxylic acid. Also, being a diol, it mixes directly with a wide range of alcohols or polyols and other performance enhancing additives with no difficulty or adverse reactions. In fact, it can be mixed in any desired proportion (to the extent that it is soluble) with any standard alcohol used in synthesizing esters, ethers, or urethane type linkages.

The yield of the above reaction of a carboxcylic acid and the preferred diol, BNPD, in the presence of either a base or acid catalyst is limited by the instability of BNPD. The yields of the desired product are significantly improved when less extreme reaction conditions are employed, especially lower temperatures. The use of activated carboxcylic acids such as acid anhydrides or acid chlorides produces a significantly higher yield and better overall quality product. The present invention also includes, but it not limited to, other forms of activated carboxcylic acids or other methods, such as the Mitsunobu reaction or the use of 2-halo-1-methylpyridinium salts, of activating the carboxcylic acid.

Esters of BNPD made via activated carboxcylic acids produce the desired esters in good yields that are low in color when worked up by standard commercially viable procedures.

While bromonitropropanediol (BNPD) is the preferred antimicrobial agent because of its proven activity and its benign effects on the environment and on humans, other alcohols, diols or polyols with bromine and nitro groups linked at the same or different carbon atoms can also be incorporated into the backbone of MWF bases. Any other antimicrobial agents that can be linked onto an alcohol reacted linkage are within the scope of the present invention.

### DESCRIPTION OF THE FIGURES

FIG. 1 shows the formation of an ester linkage with BNPD.

FIG. 2 shows the formation of an acid functional ester with BNPD.

### DETAILED DESCRIPTION OF THE INVENTION

It is well known in the art to combine alcohols with carboxylic acids to form ester linkages. One example is isopropyl oleate, the ester of isopropyl alcohol and oleic acid. Polyols are also commonly used, such as in the production of Lexolube 21-214 by Innolex. A typical ester will have the following formula: 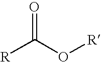
where R typically comes from the original carboxylic acid and R′ typically comes from the original alcohol. It is well known in the art that R and R′ can be the same or different. The typical example noted above as isopropyl oleate has the following structure:

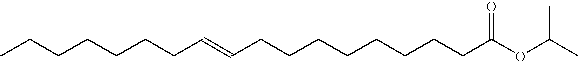

The compound bromonitropropanediol or 2-bromo-2--nitro-propane-1,3-diol (BNPD) has known antimicrobial properties. Tests on this compound have shown that it is effective against various strains of both gram positive and gram negative bacteria in concentrations of 1-50 ppm with the average minimum inhibitory concentration being around 25 ppm. In addition, work has indicated that BNPD is also antifungal. BNPD has the following structure:

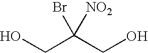

Because BNPD is a polyol, it can be combined with other alcohols, diols, or polyols in the manufacture of the esters used as MWF bases. In particular, BNPD alone or mixed with other alcohols, can be combined with activated carboxylic acids to form esters that are suitable for use as MWF bases, or as monomers in the synthesis of bromo-nitro functional polymers. This causes the active moiety to become covalently linked into the ester. In the case of the monooleate ester, the product is: 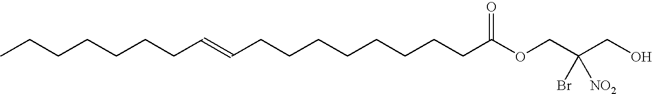
Or more generally: 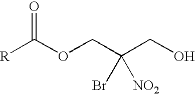
The dioleate ester can easily be made, which has the following structure: 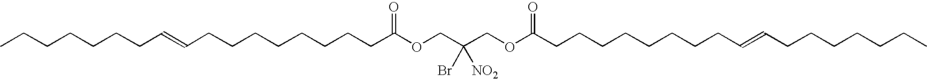
Or more generally, for the diester:

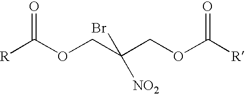

While BNPD is a preferred polyol starting point to link the active moiety into an ester MWF base, it is within the scope of the present invention to use many other materials that contain a bromine atom and nitro group linked near one another. The preferred class of compounds contains the bromine and nitro linked to the same carbon atom; however, it is felt that a moiety where the bromine and nitro are not linked to the same carbon, but near each other will still be effective. Many other similar compounds can also be used. In particular, bromonitromethanediol, bromonitroethanediol, bromonitrobutanediol, etc. can also be substituted into molecule backbones with similar results. It should be understood that these are just examples of the many compounds within the scope of the present invention. The prior art has shown that bromonitromethane is effective for the treatment of nematodes in the soil (See U.S. Pat. No. 5,013,762 which is hereby incorporated by reference) and as a general biocide (See U.S. Pat. No. 5,866,511 which is hereby incorporated by reference). It is felt that bromonitromethanediol and similar diols are equally effective.

The present invention also includes using a BNPD or BNPD analog as the terminus, such as:

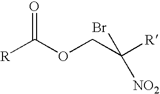

Where R′ can be, but is not limited, to CH2OH, OH, CH3, or H.

The present invention reacts BNPD or similar substituted alcohols, diols or polyols, with or without the aid of a solvent or co-solvent, with or without the aid of a catalyst, with an activated carboxylic acid or acid halide, preferably an acid chloride, to form the ester MWF base.

The present invention results in a covalently linked bromine/nitro moiety in the backbone of an ester at some frequency of occurrence that provides antibacterial or anti-fungal effects. The present invention relates to the formation of ester linkages essential to MWF bases.

As an example for a process for a making a low color, high yield BNPD diester from an acid halide, 1 to 2 molar equivalents of a fatty acid halide, preferably a chloride (such as oleyl chloride), can be placed in a vessel with around 1 molar equivalent of BNPD (a mono-ester only requires around 1 molar equivalent of the acid halide, while a diester requires are 2 molar equivalents). The vessel can be agitated and heat can be applied. The heat and agitation simply make the reaction go faster; the reactants will react without heat or agitation if left mixed. An optional vacuum can be applied to help remove the generated HX (usually HCl or HBr); this also speeds up the reaction. An inert atmosphere such as N2 helps control the color of the reaction product. After the HX stops forming, the vacuum can be removed, and a decolorizer such as carbon or similar filter aid that reduces color can be added. The preferred method is to stir for about 20 minutes; however, time is not critical. The carbon or decolorizer can then be filtered out. Diatomaceous earth can be added to reduce the filtration time.

It should be noted that with heat and vacuum, the reaction with an acid chloride can be completed within a relatively short period of time (usually <2 hrs.) by limited the heat to around 90-100 degrees C.

As an example of making an ester from an acid anhydride, around 1-2 molar equivalents of a fatty acid anhydride (1 molar equivalent for a mono-ester, 2 molar equivalents for a diester) and 1 molar equivalent of BNPD are added to a vessel. The vessel is agitated and moderate heat (90-100 degrees C.) is applied. Temperatures higher than 100 degrees C. may cause the product to take on too dark a color. The reaction can optionally be run under a vacuum or an inert atmosphere like N2. This reduces or retards undesirable color formation. The reaction vessel should be kept heated and agitated until analysis shows that the presence of alcohol groups has reached a minimum. Then a decolorizer like carbon or similar filter aid that reduces color can be added. Stirring should be continued for around 20 more minutes under the vacuum or inert atmosphere. Then the carbon or filter aid can be filtered. Diatomaceous earth can be added to reduce filtration time. For a monoester, the fatty ester formed from the reaction can be removed via techniques known in the art like extraction, filtration or distillation if the boiling point is low enough. If the anhydride is a cyclic anhydride, no purification is usually needed and a functional ester is formed.

In both the case of the acid halide and the case of the anhydride, solvents that are otherwise non-reactive can be used to solublize the BNPD or other reactants. These solvents can be vacuumed off or distilled, provided they have low enough boiling points as to not require an amount of heat that would break down the ester. A preferred solvent is methyl isobutyl ketone, or MIBK.

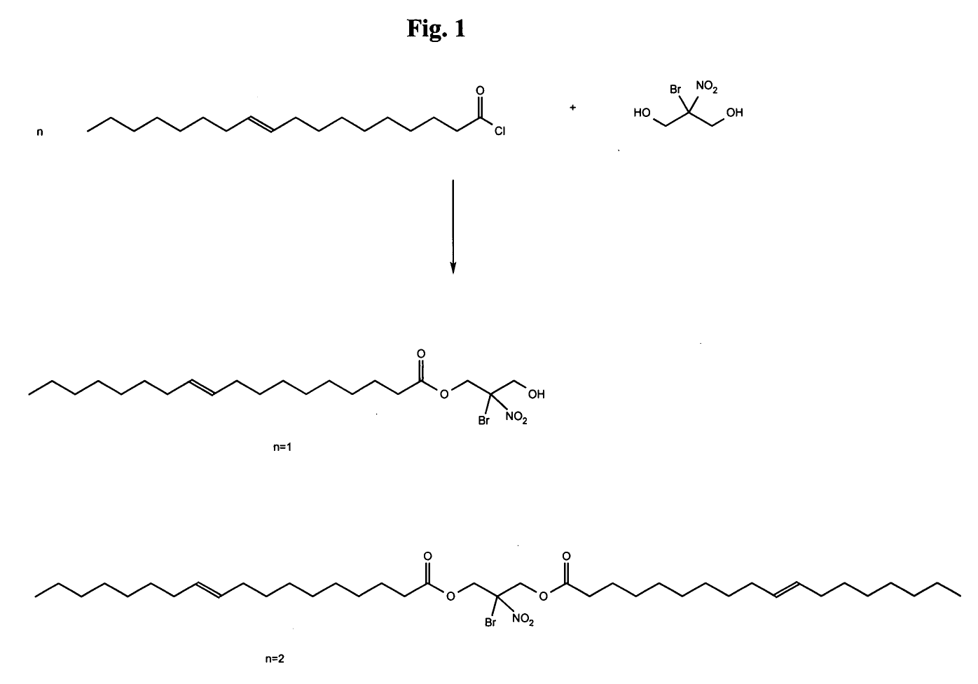

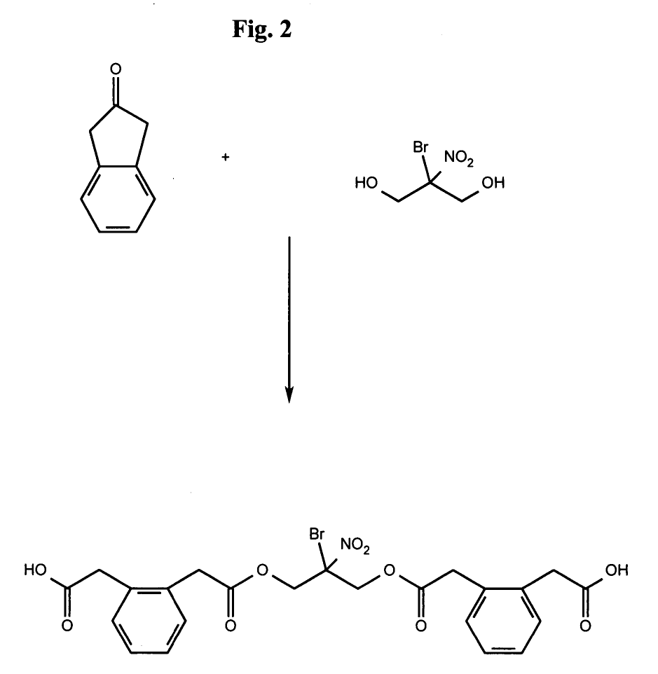

FIG. 1 shows the formation of an ester linkage with BNPD. FIG. 2 shows the formation of an acid functional ester with BNPD. Alternatively, the desired product can be made by halogenating the ester that has already been formed. For example, the dioleate ester of 2-nitropropane-1,3-diol can be bromonated with bromine gas to yield the desired product of 2-nitro-2-bromopropane-1,3-diol. The treatment of the final product with chlorine gas improves the stability and yield of the final product.

The examples and illustrations presented herein are for the purpose of understanding the concepts of the present invention. It will be clear to one with ordinary skill in the art that many other examples and structures are within the scope of the present invention. This applies particularly to classes of linkages where an example of one particular structure has been given; it will be appreciated by one skilled in the art that in such a case, the entire class of compound is within the scope of the present invention.

### EXAMPLE

### Production of a Metal Working Fluid Base

To a 250 ml round bottom flask containing 20 g BNPD was added 60.2 g oleyl chloride. The flask was then put on a rotovap and heated under vacuum. The heat was gradually increased over the course of three hours until the bath was at a full boil and no further HCl gas evolved from the vessel.

To the vessel was added 5 g of decolorizing carbon and agitated on a magnetic stirrer under vacuum for 25 minutes. The mixture was then vacuumed filtered through diatomaceous earth. The diatomaceous earth was washed with acetone and the filtered fractions combined and rotovapped.

71 g of a light yellow/light red clear, translucent liquid was recovered. Bromine analysis via x-ray fluorescence yielded 130,000 ppm bromine, versus a theoretical 109,740 ppm. FTIR showed a distinctive ester stretch.

I claim:

1. A process for making a bio-resistant ester comprising the steps of:
(a) adding around 1-2 molar equivalents of a fatty acid halide to a vessel;
(b) adding around 1 molar equivalent of BNPD;
(c) adding a sufficient amount of heat to initiate and sustain a reaction;
(d) agitating said vessel

2. The process of claim 1 further comprising the steps of:
(f) adding a decolorizer after HX ceases to be produced;
(g) allowing a sufficient time to decolorize;
(h) filtering out said decolorizer.

3. The process of claim 1 further comprising applying a vacuum after step (c).

4. The process of claim 1 further comprising removing, or allowing HX to escape the vessel as it is produced after step (c), where X is a halogen

5. The process of claim 1 further comprising adding diatomaceous earth, or other filtration aid, after step (f) or (g).

6. The process of claim 1 wherein said acid halide is an acid chloride.

7. A process for making a bio-resistant ester comprising the steps of:
(a) adding around 1-2 molar equivalents of a fatty acid anhydride to a vessel;
(b) adding around 1 molar equivalent of BNPD;
(c) adding a sufficient amount of heat to initiate and sustain a reaction;
(d) agitating said vessel;
(e) continuing said agitation and heat until the presence of alcohol groups has reached a minimum.

8. The process of claim 7 further comprising the steps of:
(f) adding a decolorizer after the presence of alcohol groups has reached a minumum;
(g) allowing a sufficient time to decolorize;
(h) filtering out said decolorizer.

9. The process of claim 7 further comprising adding diatomaceous earth, or other filtration aid, after step (f) or (g).

10. The process of claim 7 further comprising the dissolving of either or both reactants in steps (a) and (b) in a suitable solvent prior to addition to the vessel.

11. A process for making a bio-resistant ester comprising the steps of:
(a) adding around 1-2 molar equivalents of a fatty acid chloride to a vessel:
(b) adding around 1 molar equivalent of BNPD;
(c) adding a sufficient amount of heat to initiate and sustain a reaction:
(d) applying a vacuum to the reaction;
(e) agitating said vessel;
(f) removing HCl as it is produced;
(g) adding a decolorizer after HCl ceases to be produced;
(h) allowing a sufficient time to decolorize;
(i) filtering out said decolorizer.

12. The process of claim 11 wherein the acid chloride is oleyl chloride.

13. The process of claim 11 further comprising adding diatomaceous earth, or other filtration aid, after step (g) or (h).

14. A process for making a bio-resistant ester comprising the steps of:
(a) Adding around 1 molar equivalent of a nitro ester containing at least one hydrogen atom on the carbon bound to the nitrogen of the nitro group or near the carbon bound to the nitrogen of the nitro group.
(b) Adding X₂ gas under sufficient pressure, where X is a halogen and providing sufficient contact, via sparger, agitatation, or other method to add the halide to the carbon atom bound to the nitrogen of the nitro group or carbon atom near the carbon bound to the nitrogen of the nitro group.

15. The process of claim 14 further comprising the steps of:
(c) adding a decolorizing agent to allow sufficient contact, via agitation or other method, after X₂ ceases to be added;
(d) allowing a sufficient time to decolorize;
(e) filtering out said decolorizer.

16. The process of claim 14 further comprising the addition of a sufficient amount of heat to initiate and sustain a reaction.

17. The process of claim 14 further comprising the addition of a halogen gas other than that utilized in step (b), after X₂ ceases to be added.

18. The process of claim 14 further comprising adding diatomaceous earth after step (d) or (e).

19. A process for making a bio-resistant ester comprising the steps of:
(a) Adding around 1 molar equivalent of a nitro ester containing at least one hydrogen atom on the carbon bound to the nitrogen of the nitro group or near the carbon bound to the nitrogen of the nitro group.
(b) Adding Br₂ gas under sufficient pressure and providing sufficient contact, via sparger, agitatation, or other method to add the bromine to the carbon atom bound to the nitrogen of the nitro group or carbon atom near the carbon bound to the nitrogen of the nitro group.

21. The process of claim 19 further comprising the steps of:
(c) adding a decolorizing agent in such a way to allow sufficient contact, via agitation or other method, after X ceases to be added;
(d) allowing a sufficient time to decolorize;
(e) filtering out said decolorizer.

22. The process of claim 19 further comprising the addition of a sufficient amount of heat to sustain a reaction.

23. The process of claim 19 further comprising addition of Cl₂ after step (b), after Br ceases to be added.

24. The process of claim 14 further comprising adding diatomaceous earth after step (d) or (e).
